(19) United States
(12) Patent Application Publication
(10) Pub. No.: US 2026/0130773 A1
(43) Pub. Date: May 14, 2026

(54) Meta-Diaphyseal Cones for Revision Knee Arthroplasty

(71) Applicant: Mayo Foundation for Medical Education and Research, Rochester, MN (US)

(72) Inventors: John W. Sperling, Rochester, MN (US); Tad M. Mabry, Rochester, MN (US)

(21) Appl. No.: 19/384,490

(22) Filed: Nov. 10, 2025

Related U.S. Application Data
(60) Provisional application No. 63/719,307, filed on Nov. 12, 2024.

(51) Int. Cl. A61F 2/46; A61F 2/30; A61F 2/38

(52) U.S. Cl. CPC A61F 2/461; A61F 2/30771; A61F 2/3859; A61F 2002/30011; A61F 2002/3021; A61F 2002/30878

(57) ABSTRACT

A prosthetic system comprises a support structure for use in implanting a component of an implant in the first bone of the subject. The support structure (e.g., a cone) has a first end surface, a second end surface, an exterior surface extending from the first end surface to the second end surface, and an inner surface defining a passageway extending from the first end surface to the second end surface. The exterior surface of the support structure is configured to be received in a cavity of the first bone such that the first end surface is flush with or beneath a surface of the first bone, and the exterior surface of the support structure is configured to be received in the cavity of the first bone such that a section of the exterior surface of the support structure contacts cortical bone in the diaphyseal zone of the first bone

### CROSS-REFERENCES TO RELATED APPLICATIONS

This application is based on, claims benefit of, and claims priority to U.S. patent application Ser. No. 63/719,307 filed on Nov. 12, 2024, which is hereby incorporated by reference herein in its entirety for all purposes.

### STATEMENT REGARDING FEDERALLY SPONSORED RESEARCH

Not Applicable.

### BACKGROUND OF THE INVENTION

### 1. Field of the Invention

The invention relates to a prosthetic system for providing motion between a first bone and a second bone of a joint of a subject, and more particularly to revision total knee arthroplasty components that maximize contact of the component(s) with native bone.

### 2. Description of the Related Art

The number of revision total knee arthroplasty (TKA) surgeries is projected to increase significantly with an estimated 268,200 cases per year in the United States alone by 2030. A central goal of revision knee arthroplasty is to achieve excellent initial stability which is essential to obtain osseointegration for long-term survival. Achieving this goal is complicated by the frequently encountered significant bone loss present when performing a revision knee arthroplasty.

For moderate size metaphyseal defects, porous metaphyseal sleeves and cones can provide a versatile reconstruction option that achieves immediate stability with an increased potential for osseointegration. Porous metaphyseal cones may be used in conjunction with stemmed implants of numerous manufacturers and have excellent midterm survival. However, there are limited options when tibial bone loss is more extensive. Previously, the use of allografts was attempted to address these larger bone deficiencies. Preparation of an allograft is extremely time consuming and requires contouring the graft to fit the bone defect and perform fixation to the surrounding bone; even so, incorporation has been shown to be inconsistent. Moreover, the size and volume of osteolytic lesions are often underestimated during the preoperative evaluation and grafts may not be readily available. Megaprostheses have also been attempted to address large bone deficiencies in the tibia and femur; however, these implants do not osseointegrate and are associated with a high rate of reoperation. For the tibia, megaprostheses do not accommodate the patellar tendon attachment.

Stacked cones are another potential option and have been utilized in the femur and tibia to address large bone deficiencies. However, this technique has a number of significant limitations. The use of stacked cones is time-consuming, often requires multiple systems, and imparts an unstable junction at the cone-cone interface. Rather than being one uniform construct to address the significant bone deficiency, with stacked cones, the surgeon is attempting to balance two semi-round implants on top of each other. (See FIGS. 1-8 and the description of FIGS. 1-8 below.) Alignment can be very challenging and can force stems into malposition.

One study aimed to evaluate implant survivorship, radiographic results, and clinical outcomes of revision TKAs with multiple stacked cones (see Pumford et al., “Stacked Cones for the Treatment of Massive Bone Loss in Revision Total Knee Arthroplasty: 50% Reoperation Rate at Five Years”, The Journal of Arthroplasty, Volume 40, Issue 9, Supplement 1, September 2025, pages S368-S375). In this study, for the cohort of 50 stacked cone revision TKAs, 50% of patients required a reoperation.

Therefore, there exists a significant and growing unmet need to address large meta-diaphyseal bone deficiency in revision knee arthroplasty.

### SUMMARY OF THE DISCLOSURE

The notable limitations of current revision knee components to address large meta-diaphyseal bone defects resulted in a comprehensive investigation by the inventors to address this problem. The inventors have previously developed a technique for the design of tibial and femoral cones and sleeves in knee arthroplasty that maximizes device contact with native bone and minimizes risk of fracture. See PCT International Patent Application Publication No. WO 2023/141437 A1. The inventors'methodology resulted in a deep understanding of the knee anatomy and developed intuitive instrumentation and implants that can greatly facilitate revision knee arthroplasty through the use of CT scan data and 3D modeling. This methodology describes the interaction of anatomical features of the proximal tibia and distal femur and how these features change based on the specific location in the bone. Additionally, the methodology has demonstrated that the shape of the proximal tibia and distal femoral regions are quite side-specific. The present disclosure builds on this methodology and has resulted in an intuitive, streamlined workflow that can greatly improve the surgeon experience and improve patient care for patients with significant tibial bone loss. The inventors have created asymmetric and symmetric tibial metadiaphyseal cones that extend into the diaphyseal region to maximize contact with the native tibial bone. While demonstrating this for the tibia, this same technique can be used for the design of meta-diaphyseal cones for the distal femur.

In one aspect, the present disclosure provides a prosthetic system for providing motion between a first bone and a second bone of a joint of a subject wherein the first bone has a metaphyseal zone and a diaphyseal zone. The prosthetic system comprises a support structure for use in implanting a component of an implant in the first bone of the subject. The support structure has a first end surface, a second end surface, an exterior surface extending from the first end surface to the second end surface, and an inner surface defining a passageway extending from the first end surface to the second end surface. The exterior surface of the support structure is configured to be received in a cavity of the first bone such that the first end surface is flush with or beneath a surface of the first bone, and the exterior surface of the support structure is configured to be received in the cavity of the first bone such that a section of the exterior surface of the support structure contacts cortical bone in the diaphyseal zone of the first bone.

In one embodiment of the prosthetic system, the component includes a body and a stem extending away from the body, and the stem is positioned within the passageway of the support structure. In one embodiment of the prosthetic system, the first bone is the tibia, and the joint is the knee. In one embodiment of the prosthetic system, the component comprises a tibial component having a body and a stem extending away from the body, wherein the stem is positioned within the passageway of the support structure. In one embodiment of the prosthetic system, the body of the tibial component is a tibial tray. In one embodiment, the prosthetic system further comprises a tibial bearing in contact with the tibial tray, the tibial bearing having a bearing surface for articulating with the articulating surfaces of a femoral component.

In one embodiment of the prosthetic system, the first bone is the femur, and the joint is the knee. In one embodiment of the prosthetic system, the component comprises a femoral component having medial and lateral condyles with curved articulating surfaces. In one embodiment, the prosthetic system further comprises a tibial tray in contact with a tibial bearing having a bearing surface for articulating with the articulating surfaces of the femoral component.

In one embodiment of the prosthetic system, the exterior surface of the support structure is smooth. In one embodiment of the prosthetic system, the exterior surface of the support structure is roughened. In one embodiment of the prosthetic system, the exterior surface of the support structure comprises a porous ingrowth material.

In one embodiment of the prosthetic system, the support structure has a wall between the exterior surface and the inner surface, and the wall includes a slot extending away from the first end surface. In one embodiment of the prosthetic system, the component includes a body and a stem extending away from the body, and the stem is positioned within the passageway of the support structure such that a keel of the component is positioned within the slot.

In one embodiment of the prosthetic system, the support structure has a wall between the exterior surface and the inner surface, and the wall includes a pair of slots extending away from the first end surface. In one embodiment of the prosthetic system, the component includes a body and a stem extending away from the body, and the stem is positioned within the passageway of the support structure such that a first keel of the component is positioned within one of the pair of slots and a second keel of the component is positioned within another of the pair of slots.

In one embodiment of the prosthetic system, the component includes a body and a stem extending away from the body, and the stem is positioned within the passageway of the support structure such that an end of the stem opposite the body extends beyond the second end surface of the support structure.

In one embodiment of the prosthetic system, a portion of the exterior surface of the support structure has a frustoconical shape. In one embodiment of the prosthetic system, an entirety of the exterior surface of the support structure has a frustoconical shape.

In one embodiment of the prosthetic system, a longitudinal length of the support structure from the first end surface to the second end surface is in a range of 65 millimeters to 105 millimeters. In one embodiment of the prosthetic system, a transverse length of an outside diameter at the first end surface of the support structure is in a range of 25 millimeters to 45 millimeters. In one embodiment of the prosthetic system, a transverse length of an outside diameter at the second end surface of the support structure is in a range of 15 millimeters to 27 millimeters.

In one embodiment of the prosthetic system, the exterior surface of the support structure is configured to be received in the cavity of the first bone such that another section of the exterior surface of the support structure contacts cortical bone in the metaphyseal zone of the first bone. In one embodiment of the prosthetic system, the first end surface is within an axial plane defined by an outermost edge of the first end surface, and a longitudinal axis of the passageway of the support structure is offset with respect to a geometric center point of the axial plane.

In one embodiment of the prosthetic system, the support structure has a wall between the exterior surface and the inner surface, and the wall includes opposed lobes that terminate at the first end surface of the support structure.

In another aspect, the present disclosure provides a prosthetic system for providing motion between a first bone and a second bone of a joint of a subject wherein the first bone has a metaphyseal zone and a diaphyseal zone. The prosthetic system comprises a support structure for use in implanting a component of an implant in the first bone of the subject, the support structure having a first end surface, a second end surface, an exterior surface extending from the first end surface to the second end surface, and an inner surface defining a passageway extending from the first end surface to the second end surface, wherein the exterior surface of the support structure is configured to be received in a cavity of the first bone such that the first end surface is flush with or beneath a surface of the first bone, wherein the exterior surface of the support structure is configured to be received in the cavity of the first bone such that a section of the exterior surface of the support structure contacts cortical bone in the diaphyseal zone of the first bone, and wherein the support structure has bilateral symmetry with respect to a longitudinal plane containing a longitudinal axis of the passageway. In one embodiment of the prosthetic system, the component includes a body and a stem extending away from the body, and the stem is positioned within the passageway of the support structure.

In one embodiment of the prosthetic system, the first bone is the tibia, and the joint is the knee. In one embodiment of the prosthetic system, the component comprises a tibial component having a body and a stem extending away from the body, wherein the stem is positioned within the passageway of the support structure. In one embodiment of the prosthetic system, the body of the tibial component is a tibial tray. In one embodiment, the prosthetic system further comprises a tibial bearing in contact with the tibial tray, the tibial bearing having a bearing surface for articulating with the articulating surfaces of a femoral component.

In one embodiment of the prosthetic system, the first bone is the femur, and the joint is the knee. In one embodiment of the prosthetic system, the component comprises a femoral component having medial and lateral condyles with curved articulating surfaces. In one embodiment, the prosthetic system further comprises a tibial tray in contact with a tibial bearing having a bearing surface for articulating with the articulating surfaces of the femoral component.

In one embodiment of the prosthetic system, the exterior surface of the support structure is smooth. In one embodiment of the prosthetic system, the exterior surface of the support structure is roughened. In one embodiment of the prosthetic system, the exterior surface of the support structure comprises a porous ingrowth material.

In one embodiment of the prosthetic system, the support structure has a wall between the exterior surface and the inner surface, and the wall includes a slot extending away from the first end surface. In one embodiment of the prosthetic system, the component includes a body and a stem extending away from the body, and the stem is positioned within the passageway of the support structure such that a keel of the component is positioned within the slot.

In one embodiment of the prosthetic system, the support structure has a wall between the exterior surface and the inner surface, and the wall includes a pair of slots extending away from the first end surface. In one embodiment of the prosthetic system, the component includes a body and a stem extending away from the body, and the stem is positioned within the passageway of the support structure such that a first keel of the component is positioned within one of the pair of slots and a second keel of the component is positioned within another of the pair of slots.

In one embodiment of the prosthetic system, the component includes a body and a stem extending away from the body, and the stem is positioned within the passageway of the support structure such that an end of the stem opposite the body extends beyond the second end surface of the support structure.

In one embodiment of the prosthetic system, a portion of the exterior surface of the support structure has a frustoconical shape. In one embodiment of the prosthetic system, an entirety of the exterior surface of the support structure has a frustoconical shape.

In one embodiment of the prosthetic system, a longitudinal length of the support structure from the first end surface to the second end surface is in a range of 65 millimeters to 105 millimeters. In one embodiment of the prosthetic system, a transverse length of an outside diameter at the first end surface of the support structure is in a range of 25 millimeters to 45 millimeters. In one embodiment of the prosthetic system, a transverse length of an outside diameter at the second end surface of the support structure is in a range of 15 millimeters to 27 millimeters.

In one embodiment of the prosthetic system, the exterior surface of the support structure is configured to be received in the cavity of the first bone such that another section of the exterior surface of the support structure contacts cortical bone in the metaphyseal zone of the first bone.

In yet another aspect, the present disclosure provides a prosthetic system for providing motion between a first bone and a second bone of a joint of a subject wherein the first bone has a metaphyseal zone and a diaphyseal zone. The system comprises a support structure for use in implanting a component of an implant in the first bone of the subject, the support structure having a first end surface, a second end surface, an exterior surface extending from the first end surface to the second end surface, and an inner surface defining a passageway extending from the first end surface to the second end surface, wherein the exterior surface of the support structure is configured to be received in a cavity of the first bone such that the first end surface is flush with or beneath a surface of the first bone, wherein the exterior surface of the support structure is configured to be received in the cavity of the first bone such that a section of the exterior surface of the support structure contacts cortical bone in the diaphyseal zone of the first bone, and the support structure does not have bilateral symmetry with respect to a longitudinal plane containing a longitudinal axis of the passageway. In one embodiment of the prosthetic system, the component includes a body and a stem extending away from the body, and the stem is positioned within the passageway of the support structure.

In one embodiment of the prosthetic system, the first bone is the tibia, and the joint is the knee. In one embodiment of the prosthetic system, the component comprises a tibial component having a body and a stem extending away from the body, wherein the stem is positioned within the passageway of the support structure. In one embodiment of the prosthetic system, the body of the tibial component is a tibial tray. In one embodiment, the prosthetic system further comprises a tibial bearing in contact with the tibial tray, the tibial bearing having a bearing surface for articulating with the articulating surfaces of a femoral component.

In one embodiment of the prosthetic system, the first bone is the femur, and the joint is the knee. In one embodiment of the prosthetic system, the component comprises a femoral component having medial and lateral condyles with curved articulating surfaces. In one embodiment, the prosthetic system further comprises a tibial tray in contact with a tibial bearing having a bearing surface for articulating with the articulating surfaces of the femoral component.

In one embodiment of the prosthetic system, the exterior surface of the support structure is smooth. In one embodiment of the prosthetic system, the exterior surface of the support structure is roughened. In one embodiment of the prosthetic system, the exterior surface of the support structure comprises a porous ingrowth material.

In one embodiment of the prosthetic system, the component includes a body and a stem extending away from the body, and the stem is positioned within the passageway of the support structure such that an end of the stem opposite the body extends beyond the second end surface of the support structure.

In one embodiment of the prosthetic system, a portion of the exterior surface of the support structure has a frustoconical shape.

In one embodiment of the prosthetic system, the exterior surface of the support structure is configured to be received in the cavity of the first bone such that another section of the exterior surface of the support structure contacts cortical bone in the metaphyseal zone of the first bone.

In one embodiment of the prosthetic system, a longitudinal length of the support structure from the first end surface to the second end surface is in a range of 65 millimeters to 105 millimeters.

In one embodiment of the prosthetic system, the support structure has a wall between the exterior surface and the inner surface, and the wall includes opposed lobes that terminate at the first end surface of the support structure.

In one embodiment of the prosthetic system, one of the opposed lobes extends a first distance away from a longitudinal plane containing a longitudinal axis of the passageway, the other of the opposed lobes extends a second distance away from the longitudinal plane containing the longitudinal axis of the passageway, and the first distance is greater than the second distance. In one embodiment of the prosthetic system, the first distance is in a range of 22 millimeters to 40 millimeters. In one embodiment of the prosthetic system, the second distance is in a range of 18 millimeters to 35 millimeters.

In still another aspect, the present disclosure provides a method of fabricating a support structure for use in implanting a component of an implant in a first bone of a subject, wherein the first bone has a metaphyseal zone and a diaphyseal zone. The method comprises: (a) acquiring 3D digital image data of the first bone of the subject; (b) creating a 3D digital model of a support structure to be fabricated wherein the support structure has a first end surface, a second end surface, an exterior surface extending from the first end surface to the second end surface, and an inner surface defining a passageway extending from the first end surface to the second end surface, wherein the exterior surface of the support structure is configured to be received in a cavity of the first bone such that the first end surface is flush with or beneath a surface of the first bone, and wherein the exterior surface of the support structure is configured to be received in the cavity of the first bone such that a section of the exterior surface of the support structure contacts cortical bone in the diaphyseal zone of the first bone; and (c) fabricating the support structure from the 3D digital model using additive manufacturing. In one embodiment of the method, the method comprises fabricating the support structure using 3D printing. In one embodiment of the method, the first bone is the tibia. In one embodiment of the method, the first bone is the femur. In one embodiment of the method, the exterior surface of the support structure is configured to be received in the cavity of the first bone such that another section of the exterior surface of the support structure contacts cortical bone in the metaphyseal zone of the first bone.

In yet another aspect, the present disclosure provides an apparatus for fabricating a support structure for use in implanting a component of an implant in a first bone of a subject, wherein the first bone has a metaphyseal zone and a diaphyseal zone. The apparatus comprises: a 3D printing device; and a controller in electrical communication with the 3D printing device, the controller being configured to execute a program stored in the controller to: (i) receive a 3D digital model of the support structure to be fabricated wherein the support structure has a first end surface, a second end surface, an exterior surface extending from the first end surface to the second end surface, and an inner surface defining a passageway extending from the first end surface to the second end surface, wherein the exterior surface of the support structure is configured to be received in a cavity of the first bone such that the first end surface is flush with or beneath a surface of the first bone, and wherein the exterior surface of the support structure is configured to be received in the cavity of the first bone such that a section of the exterior surface of the support structure contacts cortical bone in the diaphyseal zone of the first bone; and (ii) send a group of signals to the 3D printing device to fabricate the support structure from the 3D digital model. In one embodiment of the apparatus, the first bone is the tibia. In one embodiment of the apparatus, the first bone is the femur. In one embodiment of the apparatus, the exterior surface of the support structure is configured to be received in the cavity of the first bone such that another section of the exterior surface of the support structure contacts cortical bone in the metaphyseal zone of the first bone.

It is an advantage of the present disclosure to provide a one piece meta-diaphyseal cone that results in a more stable construct without a gap, improved alignment, more efficient preparation, and greater ease of future revision compared to the currently used stacked cones. This represents a significant advance in revision knee reconstruction. The technology is available in both symmetric and asymmetric designs based on the preference of manufacturers and surgeons.

These and other features, aspects, and advantages of the present invention will become better understood upon consideration of the following detailed description, drawings, and appended claims.

### BRIEF DESCRIPTION OF THE DRAWINGS

FIG. 1 shows radiographs of a revision TKA with metaphyseal cones C presenting with tibial component loosening and significant bone loss L1.

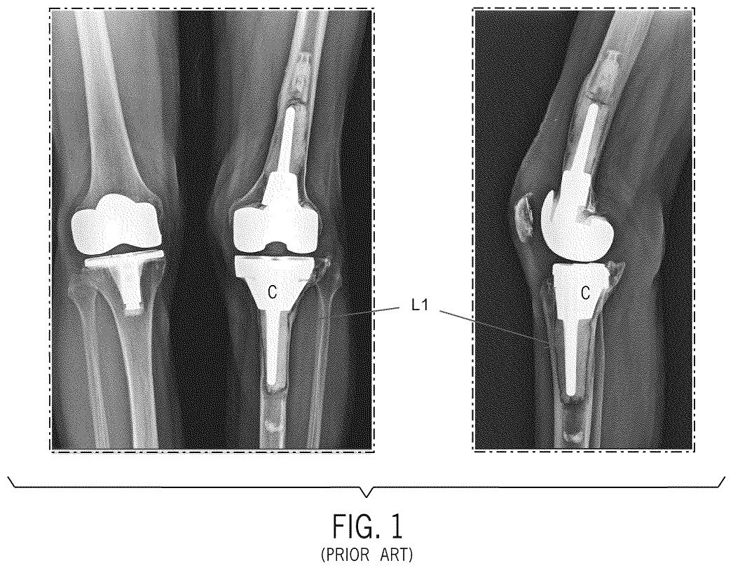

FIG. 2 shows photographs of severe cavitary and segmental tibial bone loss L2 following removal of loose tibial cone+loose tibial component.

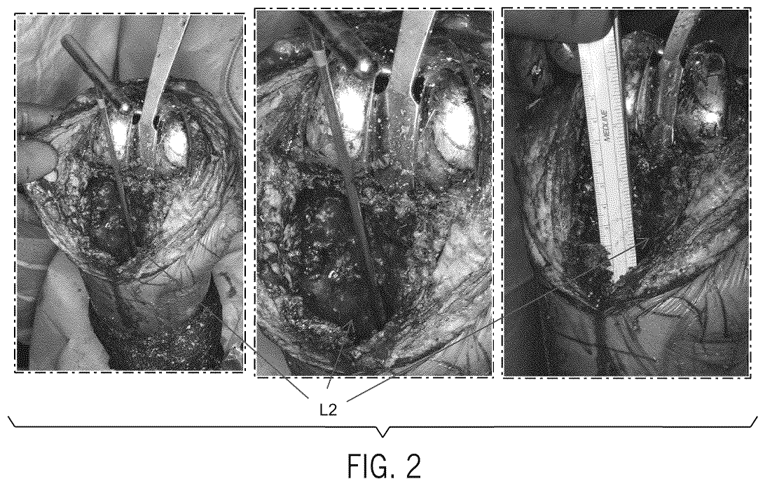

FIG. 3 shows photographs of stacked cones (top cone Ct and bottom cone Cb) attempting to reconstruct meta-diaphyseal bone loss with two separate cones Ct and Cb. Note the gaps at the cone-cone interface (Int).

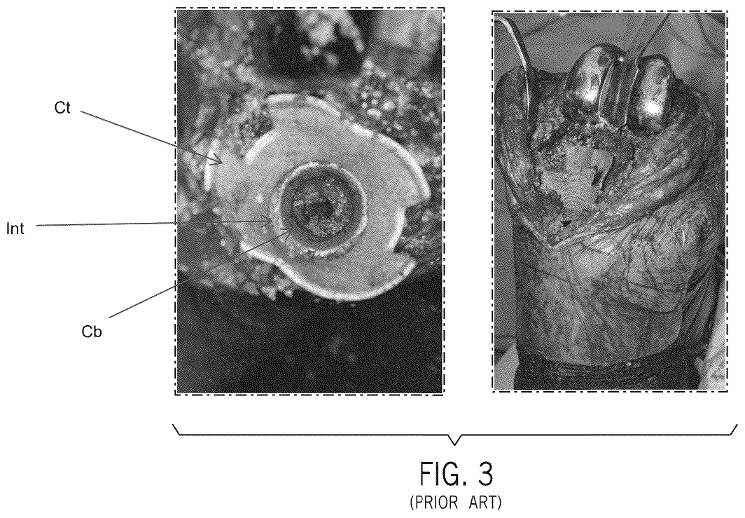

FIG. 4 shows radiographs of how stacked cones C1, C2 allow for reconstruction of a large meta-diaphyseal bone defect with substantial host bone contact. Lack of stable support between the two cones C1, C2 could compromise overall implant stability. One can see that a gap is present between the cones C1, C2 lacking uniform structural support.

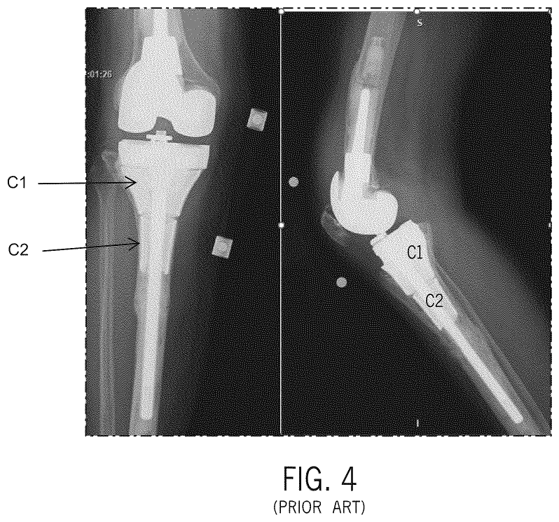

FIG. 5 shows a radiograph of loose revision tibial component with significant proximal-medial bone loss L5. Re-revision to a hinge is required to manage femur fracture nonunion and resulting cavitary+segmental tibial defect.

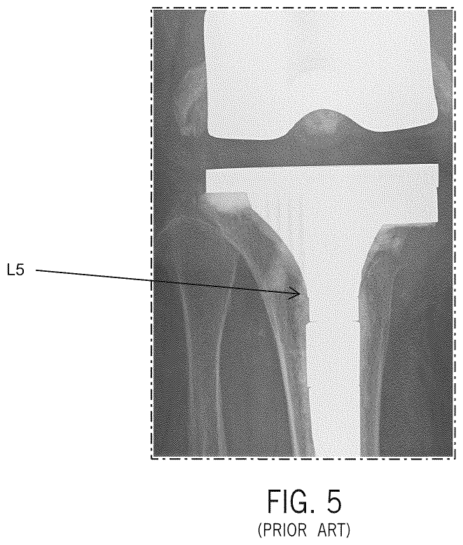

FIG. 6 shows a radiograph of stacked cone reconstruction. The upper tibial cone C6 was modified (cut) intraoperatively in order to allow appropriate tibial component position and to optimize bone contact. Again, noted is the unstable cone-cone junction J6.

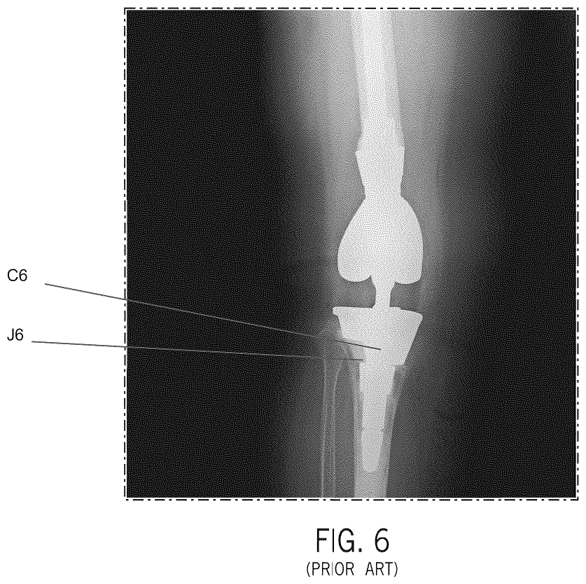

FIG. 7 shows a radiograph of loose revision tibial component with significant cavitary and segmental bone loss L7 in the setting of prior metaphyseal sleeve reconstruction.

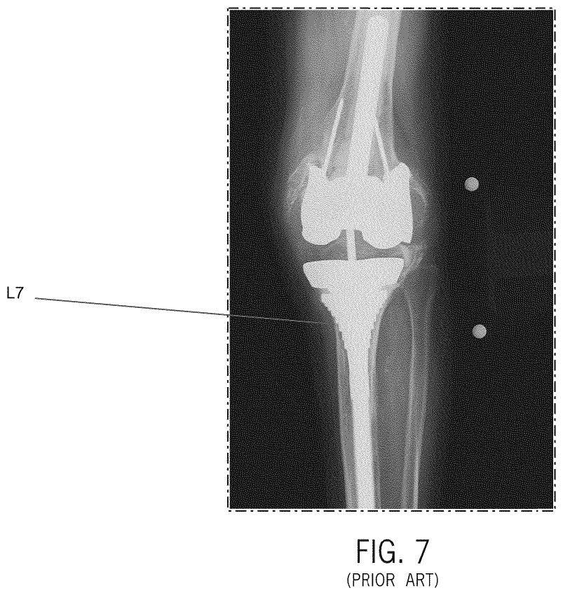

FIG. 8 shows a radiograph of stacked cones C3, C4 used to manage the large meta-diaphyseal defect. Note the gap G1 at the cone-cone interface and sagittal plane malalignment.

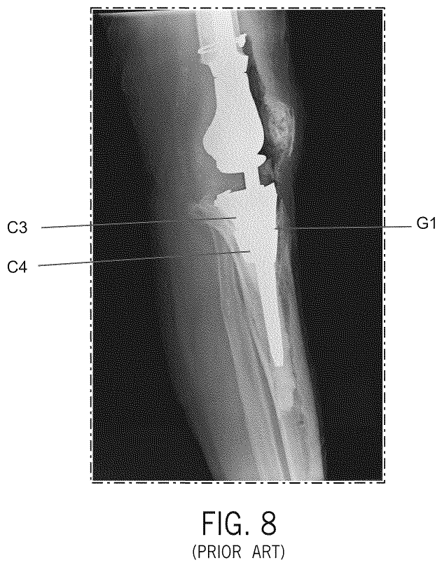

FIG. 9 shows an example symmetric meta-diaphyseal tibial cone according to one embodiment of the present disclosure wherein left is a perspective view, center is a top view, and right is a perspective view with a tibial component positioned in the symmetric meta-diaphyseal tibial cone.

FIG. 10 is a perspective view of a conical reamer designed to prepare the tibial bone for a meta-diaphyseal cone according to embodiments of the present disclosure.

FIG. 11 shows in the left panel, a side view of the symmetric meta-diaphyseal tibial cone of FIG. 9 wherein the slots in the cone accommodate keel geometry from a variety of knee replacement systems and allow rotational freedom for the example stem of the tibial component shown. The revision tibial component stem then bypasses the meta-diaphyseal cone for additional fixation in the tibial diaphysis. FIG. 11 shows in the right panel, a perspective view of the symmetric meta-diaphyseal tibial cone of FIG. 9 and the tibial component of FIG. 11 (left panel) with a tibial tray attached to the stem of the tibial component.

FIG. 12 shows a photograph wherein a circular conical reamer prepares the bone to engage the meta-diaphyseal cone. This engagement is extremely helpful to ensure osteointegration and long term fixation due to the deficiency of proximal tibial bone.

FIG. 13 shows a photograph viewed from superiorly, wherein the slots in the symmetric meta-diaphyseal tibial cone of FIG. 9 accommodate keel geometry from a variety of knee replacement systems and allow rotational freedom for a tibial component.

FIG. 14 shows photographs: (i) in the left panel, a superior view, and (ii) in the right panel, a perspective view, of a revision tibial component seated on the tibial plateau supported by a symmetrical meta-diaphyseal tibial cone of FIG. 9.

FIG. 15a shows fluoroscopy radiographs: (i) in the left panel, an anterior view, and (ii) in the right panel, a lateral view, demonstrating excellent contact of the meta-diaphyseal tibia cone of FIG. 9 with the underlying cortical bone in the tibial meta-diaphyseal region.

FIG. 15b shows radiographs: (i) in the left panel, an anterior view, of a revision tibial component seated on the tibial plateau supported by a symmetrical meta-diaphyseal tibial cone of FIG. 9, and (ii) in the right panel, an anterior view, of a prior stacked cone construction. Note the meta-diaphyseal engagement of the one-piece symmetric cone of FIG. 9 closely matches the intended reconstruction of the stacked cone construct.

FIG. 16 shows a radiograph of an anterior view of a revision tibial component seated on the tibial plateau supported by a symmetrical meta-diaphyseal tibial cone of FIG. 9 with an example of proximal bone deficiency similar to a removed metaphyseal cone or sleeve. The meta-diaphyseal tibial cone bypasses the proximal tibial bone deficiency.

FIG. 17 shows an asymmetric meta-diaphyseal tibial cone according to one embodiment of the present disclosure that is anatomic in shape and grows incrementally greater medially compared to laterally which is consistent with the underlying anatomy. In FIG. 17, left is a top view, center is a perspective view, and right is a perspective view with a tibial component positioned in the asymmetric meta-diaphyseal tibial cone.

FIG. 18 shows a perspective view of asymmetric meta-diaphyseal tibial cones according to embodiments of the present disclosure. FIG. 18 shows examples of two different sizes of asymmetric meta-diaphyseal tibial cones.

FIG. 19 shows a perspective view of broaches that are available for the asymmetric metaphyseal cones and the proximal portion of the asymmetric meta-diaphyseal cones.

FIG. 20 shows: (i) in the left panel, a perspective view of a revision tibial component having a stem and tray to supported by the asymmetrical meta-diaphyseal tibial cone of FIG. 17; and (ii) in the right panel, a perspective view of a revision tibial component stem being positioned in the asymmetrical meta-diaphyseal tibial cone of FIG. 17, wherein the stem can be used through the meta-diaphyseal cone.

FIG. 21 shows photographs wherein broaches are used for the proximal tibia and conical reamers are used for the meta-diaphyseal tibia when implanting the asymmetric meta-diaphyseal tibial cone of FIG. 17. Engagement of the remaining bone may extend far medial and lateral.

FIG. 22 shows photographs: (i) in the left panel, a superior view, and (ii) in the right panel, a perspective view of a revision tibial component seated on the tibial plateau supported by the asymmetric meta-diaphyseal tibial cone of FIG. 17.

FIG. 23a shows a radiograph of an anterior view of a revision tibial component seated on the tibial plateau supported by the asymmetric meta-diaphyseal tibial cone of FIG. 17. The proximal anatomic shape of the asymmetric meta-diaphyseal tibial cone of FIG. 17 matches the proximal tibia anatomy maximizing bone preservation and optimizing host bone contact. Note the cortical engagement of the asymmetric meta-diaphyseal tibial cone of FIG. 17 distally.

FIG. 23b shows radiographs: (i) in the left panel, an anterior view, of a revision tibial component seated on the tibial plateau supported by the asymmetric meta-diaphyseal tibial cone of FIG. 17, and (ii) in the right panel, an anterior view, of a prior stacked cone construction. Note the shape and size of the one-piece, asymmetric meta-diaphyseal cone of FIG. 17 closely matches the intended reconstruction of the stacked cone construct. There is no gap present, and the preparation is much more efficient compared to the stacked cones.

Like reference numerals will be used to refer to like parts from Figure to Figure in the following description of the drawings.

### DETAILED DESCRIPTION OF THE INVENTION

Before the present invention is described in further detail, it is to be understood that the invention is not limited to the particular embodiments described. It is also to be understood that the terminology used herein is for the purpose of describing particular embodiments only, and is not intended to be limiting. The scope of the present invention will be limited only by the claims. As used herein, the singular forms “a”, “an”, and “the” include plural embodiments unless the context clearly dictates otherwise.

It should be apparent to those skilled in the art that many additional modifications beside those described herein are possible without departing from the inventive concepts. In interpreting this disclosure, all terms should be interpreted in the broadest possible manner consistent with the context. Variations of the term “comprising”, “including”, or “having” should be interpreted as referring to elements, components, or steps in a non-exclusive manner, so the referenced elements, components, or steps may be combined with other elements, components, or steps that are not expressly referenced. Embodiments referenced as “comprising”, “including”, or “having” certain elements are also contemplated as “consisting essentially of” and “consisting of” those elements, unless the context clearly dictates otherwise. It should be appreciated that aspects of the disclosure that are described with respect to a device are applicable to the methods, and vice versa, unless the context explicitly dictates otherwise.

The present disclosure provides a prosthetic system for providing motion between a first bone and a second bone of a joint of a subject. A “subject” is a mammal, preferably a human. The first bone and the second bone can be a long bone (e.g., tibia or femur) that is composed of a hollow shaft, or diaphysis; a flared, cone-shaped metaphysis adjacent the diaphysis; and a rounded epiphysis adjacent the metaphysis opposite the diaphysis. The diaphysis is composed primarily of dense cortical bone, whereas the metaphysis and epiphysis are composed of trabecular meshwork bone surrounded by a relatively thin shell of dense cortical bone.

### Symmetric Meta-Diaphyseal Cones

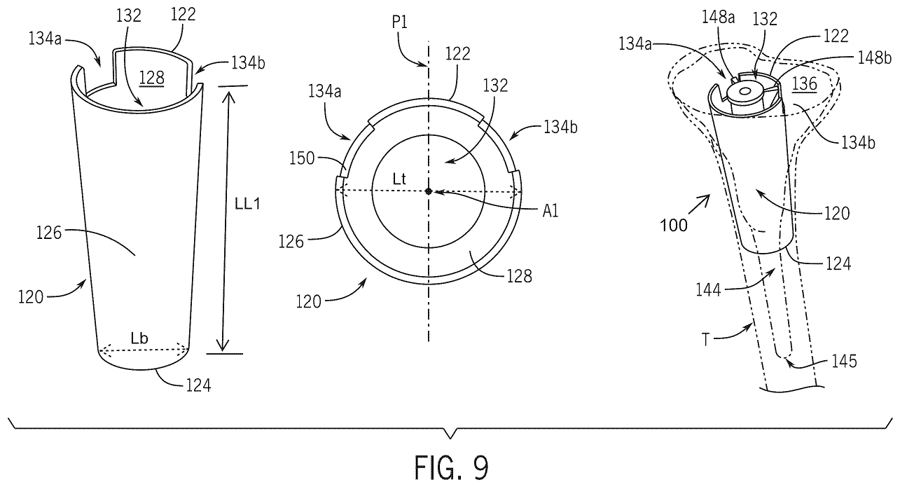

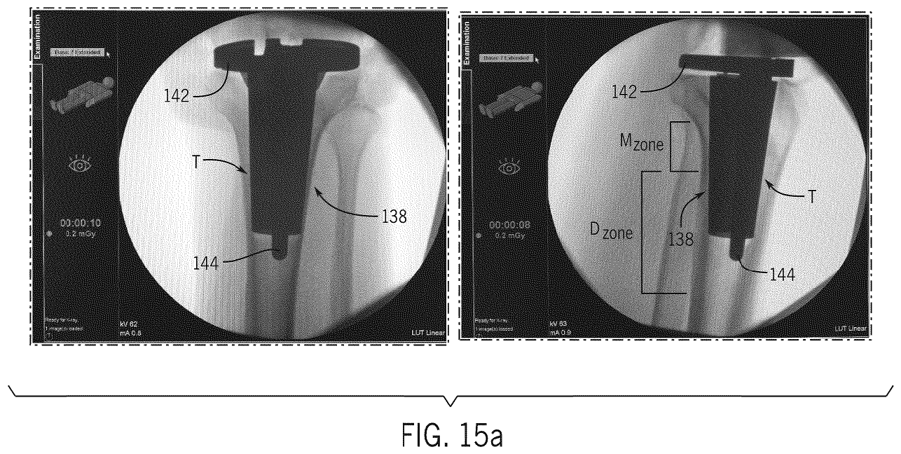

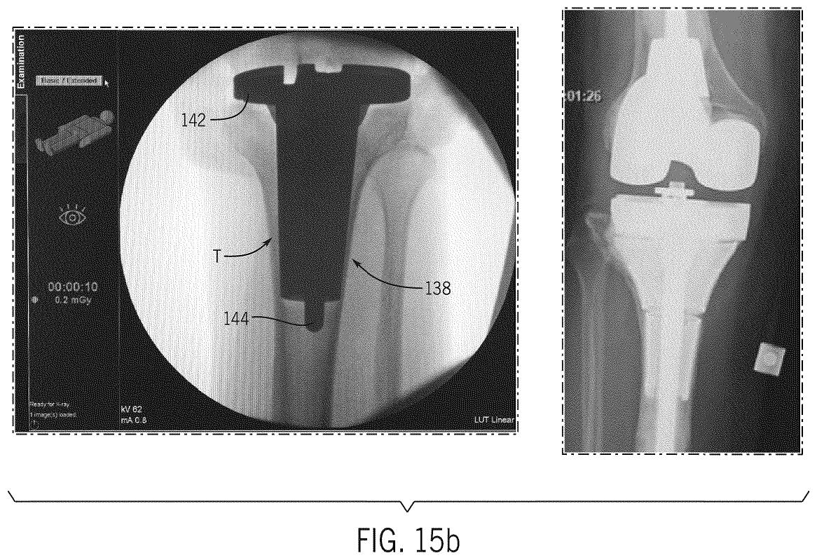

Turning now to FIGS. 9, 11, and 14-16, there is shown a non-limiting example prosthetic system 100 for providing motion between a tibia T and a femur F of a knee joint of a subject. The tibia T has a metaphyseal zone M_{zone} and a diaphyseal zone D_{zone} (see FIG. 15a). The prosthetic system 100 includes a symmetric cone 120 (i.e., a support structure) for use in implanting a tibial component of an implant in the tibia T of the subject. The cone 120 has a first end surface 122, a second end surface 124, an exterior surface 126 extending from the first end surface 122 to the second end surface 124, and an inner surface 128 defining a passageway 132 extending from the first end surface 122 to the second end surface 124. At least a portion of the exterior surface 126 of the cone 120 has a frustoconical shape. Alternatively, the entirety of the exterior surface 126 of the cone 120 can have a frustoconical shape. Looking at FIG. 9, the cone 120 has bilateral symmetry with respect to a longitudinal plane P1 containing a longitudinal axis A1 of the passageway 132 of the cone 120.

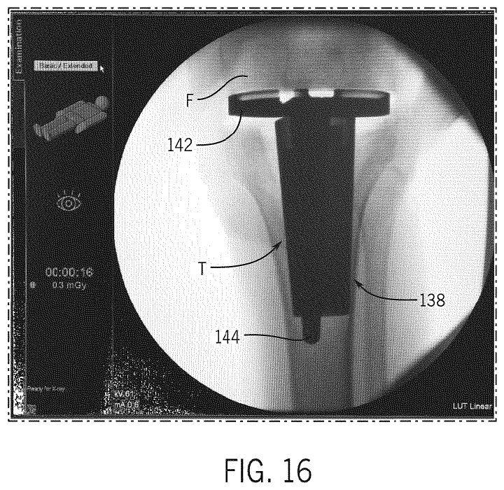

The exterior surface 126 of the cone 120 is configured to be received in a cavity of the tibia T such that the first end surface 122 is flush with or beneath a proximal surface 136 of the tibia T. The exterior surface 126 of the cone 120 is configured to be received in the cavity of the tibia T such that a section of the exterior surface 126 of the cone 120 contacts cortical bone 138 in the diaphyseal zone D_{zone} of the tibia T (see FIGS. 15a, 15b, and 16). The exterior surface 126 of the cone 120 can be configured to be received in the cavity of the tibia T such that a section of the exterior surface 126 of the cone 120 contacts cortical bone 138 in the metaphyseal zone M_{zone} of the tibia T. In one embodiment of the cone 120, a diameter range of the cone 120 can be selected that results in a minimum of 50% contact with the cortical bone or until the minimum cortical bone thickness is 1 millimeter. A longitudinal length LL1 of the cone 120 from the first end surface 122 to the second end surface 124 may be in a range of 65 millimeters to 105 millimeters, or in a range of 70 millimeters to 100 millimeters, or in a range of 75 millimeters to 95 millimeters, or in a range of 80 millimeters to 90 millimeters. A transverse length Lt of the outside diameter at the first end surface 122 of the cone 120 may be in a range of 25 millimeters to 45 millimeters, or in a range of 30 millimeters to 40 millimeters, or in a range of 32 millimeters to 38 millimeters, or in a range of 34 millimeters to 36 millimeters. A transverse length Lb of the outside diameter at the second end surface 124 of the cone 120 may be in a range of 15 millimeters to 27 millimeters, or in a range of 18 millimeters to 24 millimeters, or in a range of 19 millimeters to 23 millimeters, or in a range of 20 millimeters to 22 millimeters.

A tibial component for use in the prosthetic system 100 includes a body (tray 142) and a stem 144 extending away from the body, and the stem 144 is positioned within the passageway 132 of the cone 120. A tibial bearing can be in contact with the tibial tray 142, wherein the tibial bearing has a bearing surface for articulating with the articulating surfaces of a femoral component having medial and lateral condyles with curved articulating surfaces.

The cone 120 has a wall 150 between the exterior surface 126 and the inner surface 128. The wall includes a pair of slots 134a, 134b extending away from the first end surface 122. The stem 144 is positioned within the passageway 132 of the cone 120 such that a first keel 148a of the tibial component is positioned within the slot 134a and a second keel 148b of the tibial component is positioned within the slot 134b. The slots 134a, 134b are wide enough to provide rotational movement of the stem 144. The stem 144 is positioned within the passageway of the cone 120 such that an end 145 of the stem 144 opposite the tray 142 extends beyond the second end surface 124 of the cone 120.

### Asymmetric Meta-Diaphyseal Cones

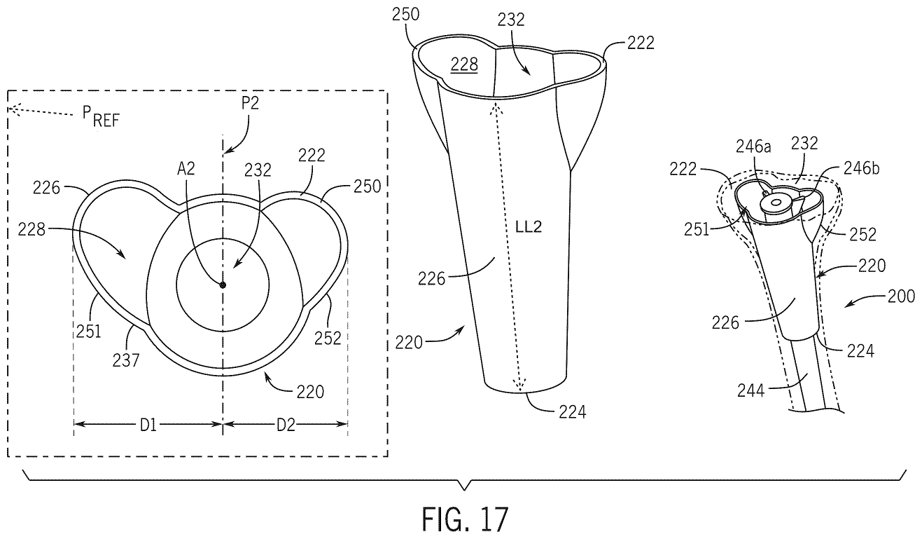

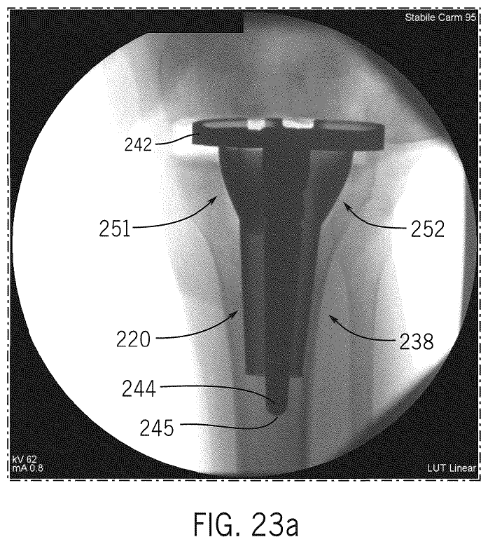

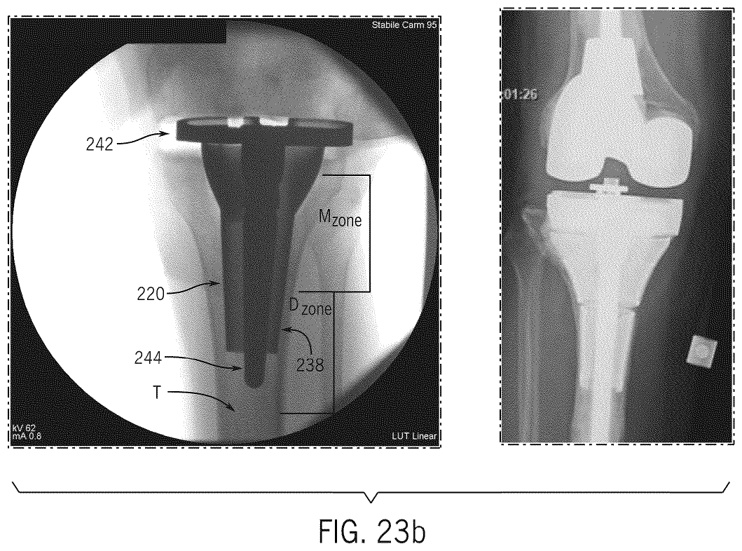

Referring now to FIGS. 17 and 20-23b, there is shown a non-limiting example prosthetic system 200 for providing motion between a tibia T and a femur F of a knee joint of a subject. The tibia T has a metaphyseal zone M_{zone} and a diaphyseal zone D_{zone} (see FIG. 23b). The prosthetic system 200 includes an asymmetric cone 220 (i.e., a support structure) for use in implanting a tibial component of an implant in the tibia T of the subject. The cone 220 has a first end surface 222, a second end surface 224, an exterior surface 226 extending from the first end surface 222 to the second end surface 224, and an inner surface 228 defining a passageway 232 extending from the first end surface 222 to the second end surface 224. At least a portion of the exterior surface 226 of the cone 220 has a frustoconical shape. Looking at FIG. 17, the first end surface 222 is within an axial plane defined by an outermost edge 237 of the first end surface 222 within a reference plane P_{ref}, and a longitudinal axis A2 of the passageway 232 of the cone 220 is offset with respect to a geometric center point of the axial plane. The cone 220 does not have bilateral symmetry with respect to a longitudinal plane P2 containing the longitudinal axis A2 of the passageway 232 of the cone 220.

The exterior surface 226 of the cone 220 is configured to be received in a cavity of the tibia T such that the first end surface 222 is flush with or beneath a proximal surface 236 of the tibia T. The exterior surface 226 of the cone 220 is configured to be received in the cavity of the tibia T such that a section of the exterior surface 226 of the cone 220 contacts cortical bone 238 in the diaphyseal zone D_{zone} of the tibia T (see FIGS. 23a, 23b). The exterior surface 226 of the cone 220 is configured to be received in the cavity of the tibia T such that a section of the exterior surface 226 of the cone 220 contacts cortical bone 238 in the metaphyseal zone M_{zone} of the tibia T.

A tibial component for use with the prosthetic system 200 includes a body (tray 242) and a stem 244 extending away from the body, and the stem 244 is positioned within the passageway 232 of the cone 220. The stem 244 can be positioned within the passageway 232 of the cone 220 such that an end 245 of the stem 244 opposite the tray 242 extends beyond the second end surface 224 of the cone 220. A tibial bearing can be in contact with the tibial tray 242, wherein the tibial bearing has a bearing surface for articulating with the articulating surfaces of a femoral component having medial and lateral condyles with curved articulating surfaces.

Looking at FIG. 17, the cone 220 has a wall 250 between the exterior surface 226 and the inner surface 228. The wall includes a pair of opposed lobes 251, 252 that terminate at the first end surface 222 of the cone 220. The lobe 251 extends a first distance D1 away from the longitudinal plane P2 containing the longitudinal axis A2 of the passageway 232, and the lobe 252 extends a second distance D2 away from the longitudinal plane P2 containing the longitudinal axis A2 of the passageway 232. In this embodiment, the length of the first distance D1 is greater than the length of the second distance D2. A longitudinal length LL2 of the cone 220 from the first end surface 222 to the second end surface 224 may be in a range of 65 millimeters to 105 millimeters, or in a range of 70 millimeters to 100 millimeters, or in a range of 75 millimeters to 95 millimeters, or in a range of 80 millimeters to 90 millimeters. The first distance D1 may be in a range of 22 millimeters to 40 millimeters, or in a range of 25 millimeters to 37 millimeters, or in a range of 27 millimeters to 35 millimeters, or in a range of 29 millimeters to 33 millimeters. The second distance D2 may be in a range of 18 millimeters to 35 millimeters, or in a range of 21 millimeters to 32 millimeters, or in a range of 23 millimeters to 30 millimeters, or in a range of 25 millimeters to 28 millimeters.

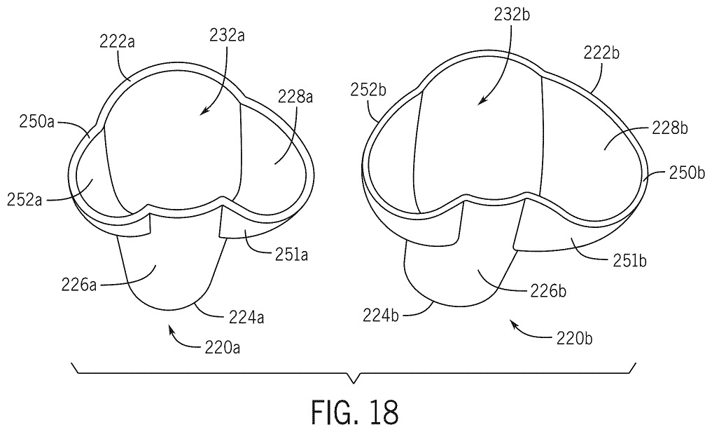

Turning to FIG. 18, non-limiting examples of different sizes of cones 220a and 220b are shown. Cone 220a has a first end surface 222a, a second end surface 224a, an exterior surface 226a extending from the first end surface 222a to the second end surface 224a, and an inner surface 228a defining a passageway 232a extending from the first end surface 222a to the second end surface 224a. At least a portion of the exterior surface 226a of the cone 220a has a frustoconical shape. The exterior surface 226a of the cone 220a is configured to be received in a cavity of the tibia T such that the first end surface 222a is flush with or beneath a proximal surface 236 of the tibia T. The exterior surface 226a of the cone 220a is configured to be received in the cavity of the tibia T such that a section of the exterior surface 226a of the cone 220a contacts cortical bone 238 in the diaphyseal zone D_{zone} of the tibia T (see FIGS. 23a, 23b). The exterior surface 226a of the cone 220a is configured to be received in the cavity of the tibia T such that a section of the exterior surface 226a of the cone 220a contacts cortical bone 238 in the metaphyseal zone M_{zone} of the tibia T. The cone 220a has a wall 250a between the exterior surface 226a and the inner surface 228a. The wall includes a pair of opposed lobes 251a, 252a that terminate at the first end surface 222a of the cone 220a. In one embodiment of the cone 220, a diameter range of the cone 220 can be selected that results in a minimum of 50% contact with the cortical bone or until the minimum cortical bone thickness is 1 millimeter.

Still referring to FIG. 18, cone 220b has a first end surface 222b, a second end surface 224b, an exterior surface 226b extending from the first end surface 222b to the second end surface 224b, and an inner surface 228b defining a passageway 232b extending from the first end surface 222b to the second end surface 224b. At least a portion of the exterior surface 226b of the cone 220b has a frustoconical shape. The exterior surface 226b of the cone 220b is configured to be received in a cavity of the tibia T such that the first end surface 222b is flush with or beneath a proximal surface 236 of the tibia T. The exterior surface 226b of the cone 220b is configured to be received in the cavity of the tibia T such that a section of the exterior surface 226b of the cone 220b contacts cortical bone 238 in the diaphyseal zone D_{zone} of the tibia T (see FIGS. 23a, 23b). The exterior surface 226b of the cone 220b is configured to be received in the cavity of the tibia T such that a section of the exterior surface 226b of the cone 220b contacts cortical bone 238 in the metaphyseal zone M_{zone} of the tibia T. The cone 220b has a wall 250b between the exterior surface 226b and the inner surface 228b. The wall includes a pair of opposed lobes 251b, 252b that terminate at the first end surface 222b of the cone 220b.

A breadth of sizes (such as cones 120, 220, 220a, 220b) greatly broadens the potential for use of cones in revision knee arthroplasty. Current cones have limited sizes with large changes between sizes forcing implants that are too small and do not maximize contact or necessitate additional bone removal to make them fit. The availability of smaller cone sizes in the invention also broadens the market for use of cones, which is a premium product with premium pricing which is attractive to manufacturers.

Additionally, the system and implants create a streamlined surgical flow. For the tibia, the distal sizing is confirmed with cylindrical reamers and then broaches for the cones are utilized to ensure maximum support and minimize bone removal. The same is true for the femur after engaging with proximal femoral diaphyseal bone. The system of the invention allows any size cone to be used with any size stem. This greatly facilitates broadening the use of cones in revision cases and expands the market opportunity. For example, a small cone can be used in any size patient providing a better option compared to bone graft, cement, or removing additional bone to make current non-anatomically shaped and sized cones fit.

The cones 120, 220, 220a, 220b and tibial components may be formed from a metal alloy such as titanium alloys (e.g., titanium-6-aluminum-4-vanadium), cobalt-chromium alloys, stainless steel alloys and tantalum alloys; nonresorbable ceramics such as aluminum oxide and zirconia; nonresorbable polymeric materials such as polyethylene; or composite materials such as carbon fiber-reinforced polymers (e.g., polysulfone). In some configurations, the cone is formed from a tantalum based porous material, or it may be made from another metal that is coated with a tantalum-based porous metal or other porous coating.

### EXAMPLE

The following Example has been presented in order to further illustrate the invention and is not intended to limit the invention in any way. The statements provided in the Example are presented without being bound by theory.

### Methodology

To demonstrate the use of the methodology of the present disclosure to develop anatomically shaped meta-diaphyseal tibial cones, two sizes of tibial cones were utilized. In the disclosure of WO 2023/141437 A1, tibias were divided into seven groups based on size. In this Example, size 3 and size 6 tibias were used to represent a mid-range size as well as a larger size tibia. The diameter of the inner cortex of the tibia was calculated 55 millimeters and 85 millimeters distal to the cut surface. The ideal diameter of a cone at these levels was determined by selecting a diameter that resulted in a minimum of 50% contact with the cortical bone or until the minimum cortical bone thickness was 1 millimeter. For the size 3 meta-diaphyseal cone, the diameter at 55 millimeters distal to the tibial cut surface was 26 millimeters and at 85 millimeters distal to the tibial cut surface it was 21 millimeters. For the size 6 meta-diaphyseal cone, the diameter at 55 millimeters distal to the tibial cut surface was 30 millimeters and at 85 millimeters distal to the tibial cut surface it was 25 millimeters. The cones were then tapered based on these measurements.

### Implants

Two different non-limiting example types of meta-diaphyseal cones are described in this Example. Symmetric Meta-Diaphyseal Cones

The inner circular diameter of the invented cones of WO 2023/141437 A1 at the most proximal end was used. The cone was then tapered distally to correspond to the inner cortical diameter at 55 mm and 85 mm from the tibial cut surface. The benefits of a symmetric cone are less inventory as it is not left and right specific and ease of revision if the cone needs to be removed. (See FIG. 9.)

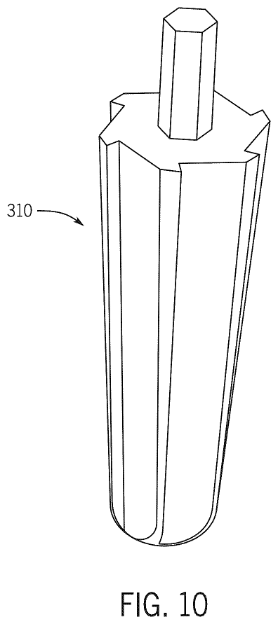

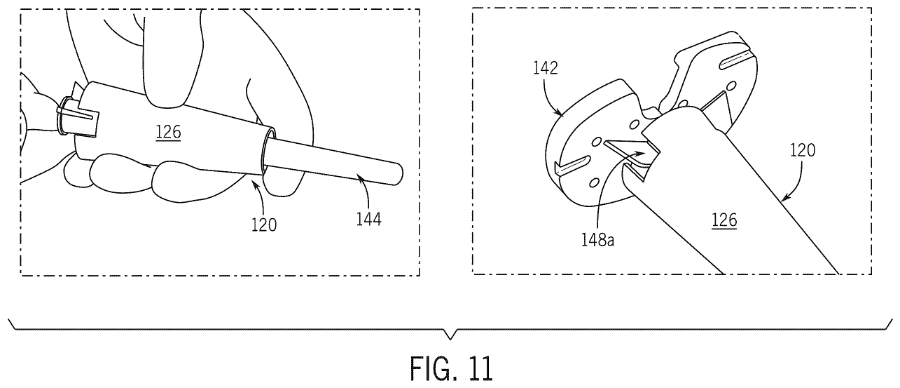

Circular conical tapered reamers were also developed that corresponded to the inner circular diameter of both the asymmetric and symmetric meta-diaphyseal cones. Use of conical circular reamers 310 is a technique that is familiar to surgeons. These circular reamers 310 facilitate preparing the cortical bone to optimize osteointegration. (See FIG. 10.) These reamers could also be cannulated as an option for ease of use. Slots 134a, 134b in the symmetric cones 120 accommodate keel 148 geometry from a variety of knee replacement systems and allow rotational freedom for a tibial component. (See FIG. 11.) A revision tibial component stem 144 may then be used to bypass the meta-diaphyseal cone 120 for additional fixation in the tibial diaphysis zone D_{zone}. (See FIGS. 11, 15a, 15b, and 16.)

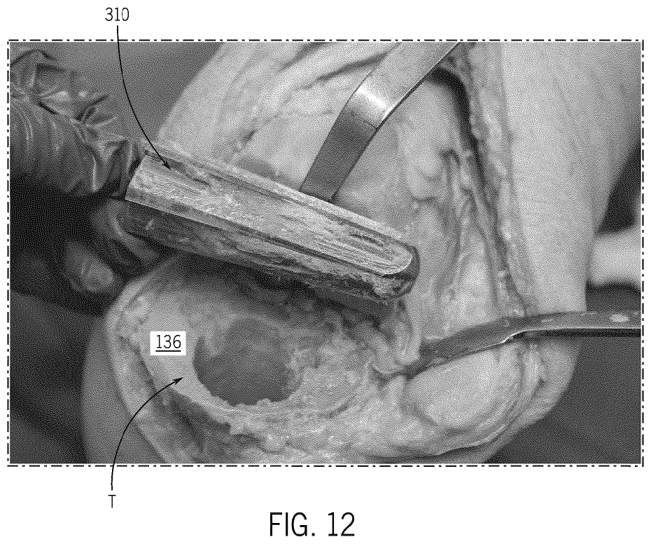

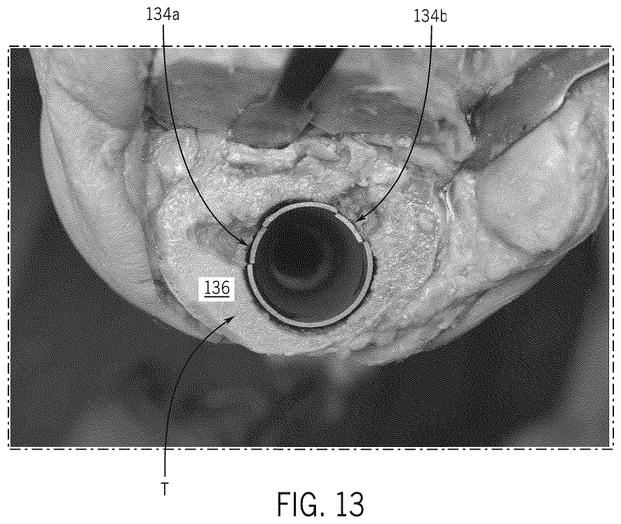

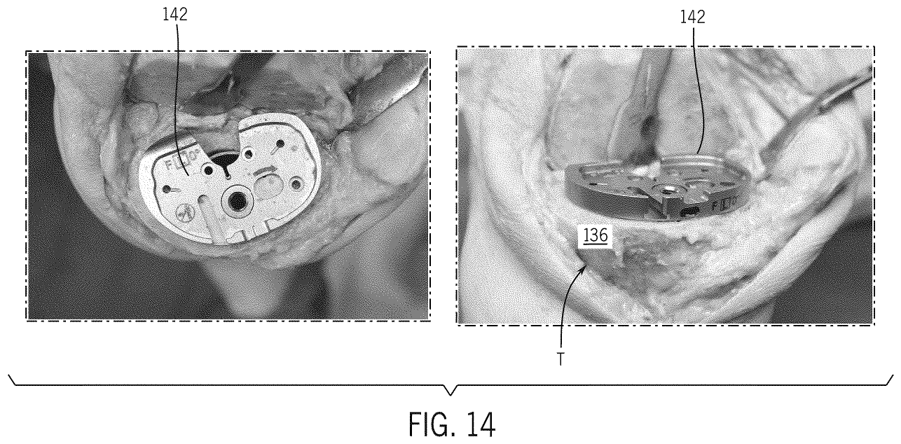

A cadaveric validation of the symmetric meta-diaphyseal cone 100 was performed. One can see how the circular conical reamers 310 prepare the cortical bone to engage with the meta-diaphyseal cone 120 and optimize the bone for osseointegration with the implant. (See FIG. 12.) The slots 134, 134b on the meta-diaphyseal cone 120 can accommodate keel 148a, 148b geometry from a wide variety of knee replacement systems and allow rotational freedom for the tibial component. (See FIG. 13.) A revision tibial component seated on the tibial plateau is supported by a symmetrical meta-diaphyseal tibial cone 120. (See FIG. 14.) One can see excellent contact of the cone with the underlying cortical bone in the tibial meta-diaphyseal region. (See FIG. 15a.) Note the meta-diaphyseal engagement of the one-piece symmetric cone 120 closely matches the intended reconstruction of the stacked cone construct (See FIG. 15b). The one piece meta-diaphyseal cone 120 by the inventors results in a more stable construct without a gap, improved alignment, more efficient preparation, and greater ease of future revision compared to the currently used stacked cones. In another example, one can see proximal bone deficiency similar to a removed metaphyseal cone or sleeve. The meta-diaphyseal cone 120 effectively bypasses the proximal tibial bone deficiency. (See FIG. 16.)

### Asymmetric Meta-Diaphyseal Cones

The designed asymmetric, right and left specific, cones of WO 2023/141437 A1 were extended distally using the diameter that corresponded to the inner cortical diameter at 55 mm and 85 mm down from the cut surface. (See FIG. 17.) The asymmetric cones 220, 220a, 220b grow incrementally greater medially compared to laterally which is consistent with the underlying anatomy. (See FIG. 17.) One can see two additional different sizes of meta-diaphyseal cones 220a, 220b. (See FIG. 18.)

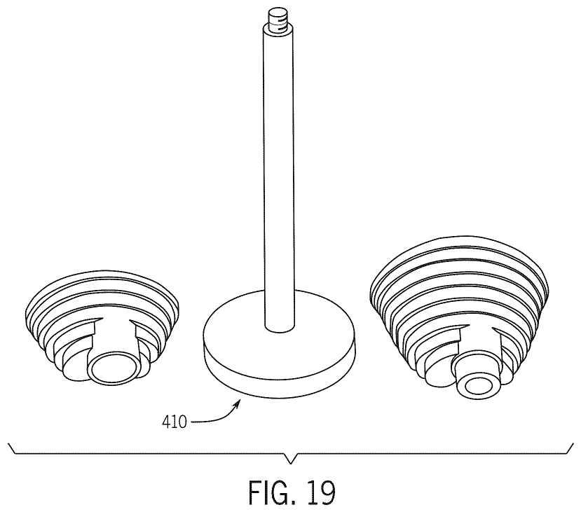

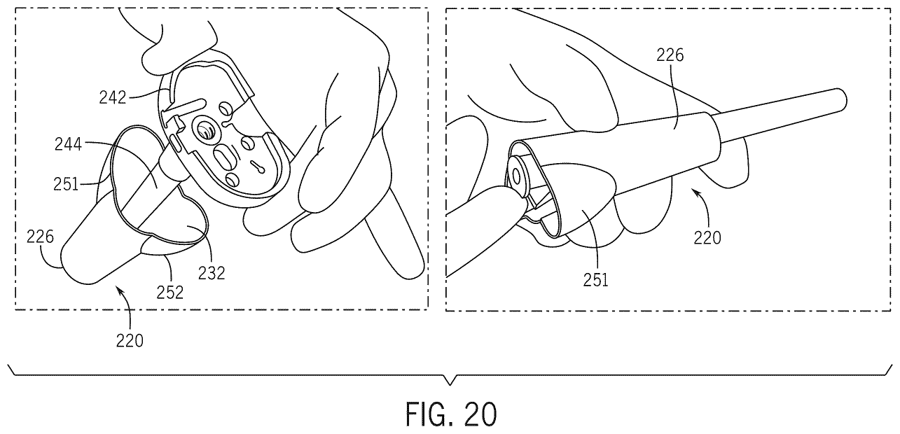

For the asymmetric cones 220, 220a, 220b, broaches 410 that were designed by the inventors for metaphyseal sleeves and cones can be used for preparation of the proximal aspect of the meta-diaphyseal cones 220, 220a, 220b. (See FIG. 19.) The conical circular reamers 310 can then be used for distal diaphyseal tibia preparation. (See FIG. 10.) A revision tibial component stem 244 can then be used to bypass the meta-diaphyseal cone 220, 220a, 220b for additional fixation in the tibial diaphysis. (See FIG. 20.)

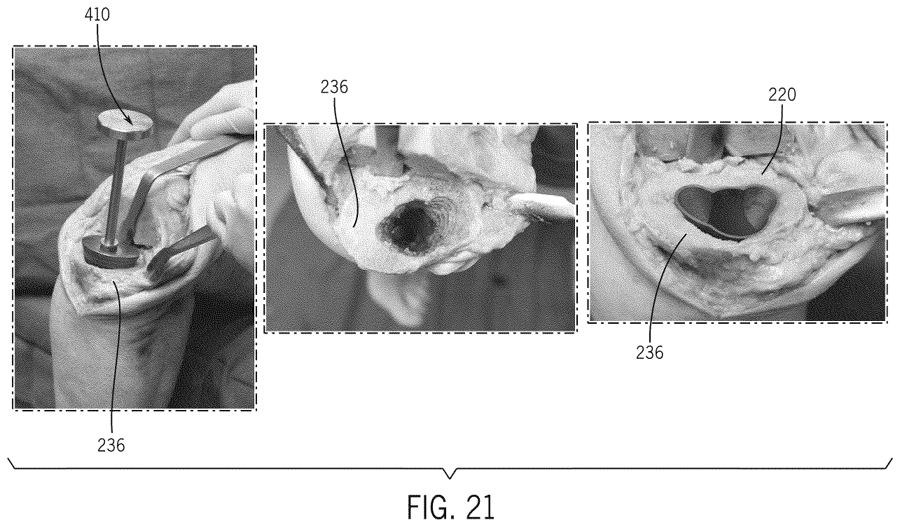

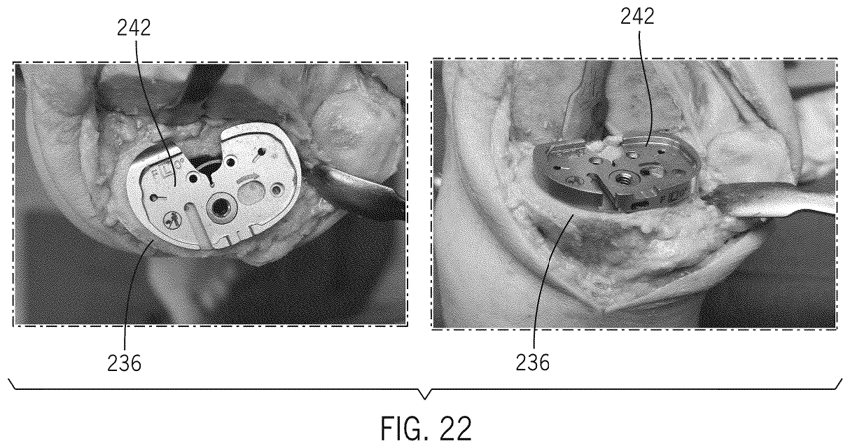

A cadaveric validation of the asymmetric meta-diaphyseal cones 220, 220a, 220b was performed. Broaches 410 are used for the proximal tibia and the circular conical reamers 310 are used for the meta-diaphyseal tibia. Engagement of the remaining bone which may extend far medial and lateral is facilitated with the asymmetric design of cones 220, 220a, 220b. (See FIG. 21.) Example of a revision tibial component seated on the tibial plateau supported by an asymmetric meta-diaphyseal tibial cone 220 is shown in FIG. 22. Proximal anatomic shape of asymmetric meta-diaphyseal cone 220 matches the proximal tibia anatomy maximizing bone preservation and optimizing host bone contact. (See FIG. 23a.) This results in excellent engagement of the distal tibial cortical bone which is essential for osseointegration and stability. Note the shape and size of the one-piece, asymmetric meta-diaphyseal cone 220 closely matches the intended reconstruction of the stacked cone construct with more efficient preparation and improved stability as well as alignment. (See FIG. 23b.)

### Method and Apparatus for Fabricating Meta-Diaphyseal Cones

Meta-diaphyseal cones of the present disclosure can be fabricated as an off the shelf support structure for use in implanting a component of an implant, as well as a custom patient specific support structure for use in implanting a component of an implant.

The invention provides a method of fabricating a support structure for use in implanting a component of an implant in a first bone of a subject, wherein the first bone has a metaphyseal zone and a diaphyseal zone. The method comprises acquiring 3D digital image data of the first bone of the subject. A scan can be taken with a computed tomography scanner to capture information on the shape and size of the first bone and to reproduce 3D models of the first bone, thus allowing complete digitalization of the first bone anatomy. The method then comprises creating a 3D digital model of a support structure to be fabricated wherein the support structure has a first end surface, a second end surface, an exterior surface extending from the first end surface to the second end surface, and an inner surface defining a passageway extending from the first end surface to the second end surface, wherein the exterior surface of the support structure is configured to be received in a cavity of the first bone such that the first end surface is flush with or beneath a surface of the first bone, and wherein the exterior surface of the support structure is configured to be received in the cavity of the first bone such that a section of the exterior surface of the support structure contacts cortical bone in the diaphyseal zone of the first bone. The method then comprises fabricating the support structure from the 3D digital model using additive manufacturing. In one embodiment, the method comprises fabricating the support structure using 3D printing. In one embodiment, the first bone is the tibia. In one embodiment, the first bone is the femur. With the advent of 3D printing, one could see how surgeons may wish to get a CT scan or other imaging modality and that the custom metaphyseal cone could be manufactured for that patient.

The invention provides an apparatus for fabricating a support structure for use in implanting a component of an implant in a first bone of a subject, wherein the first bone has a metaphyseal zone and a diaphyseal zone. The apparatus comprises: a 3D printing device; and a controller in electrical communication with the 3D printing device, the controller being configured to execute a program stored in the controller to: (i) receive a 3D digital model of the support structure to be fabricated wherein the support structure has a first end surface, a second end surface, an exterior surface extending from the first end surface to the second end surface, and an inner surface defining a passageway extending from the first end surface to the second end surface, wherein the exterior surface of the support structure is configured to be received in a cavity of the first bone such that the first end surface is flush with or beneath a surface of the first bone, and wherein the exterior surface of the support structure is configured to be received in the cavity of the first bone such that a section of the exterior surface of the support structure contacts cortical bone in the diaphyseal zone of the first bone; and (ii) send a group of signals to the 3D printing device to fabricate the support structure from the 3D digital model. In one embodiment, the first bone is the tibia. In one embodiment, the first bone is the femur.

Thus, the invention provides a one piece meta-diaphyseal cone that results in a more stable construct without a gap, improved alignment, more efficient preparation, and greater ease of future revision compared to the currently used stacked cones. This represents a significant advance in revision knee reconstruction. The technology is available in both symmetric and asymmetric designs based on the preference of manufacturers and surgeons.

In light of the principles and example embodiments described and illustrated herein, it will be recognized that the example embodiments can be modified in arrangement and detail without departing from such principles. Also, the foregoing discussion has focused on particular embodiments, but other configurations are also contemplated. In particular, even though expressions such as “in one embodiment”, “in another embodiment,” or the like are used herein, these phrases are meant to generally reference embodiment possibilities, and are not intended to limit the invention to particular embodiment configurations. As used herein, these terms may reference the same or different embodiments that are combinable into other embodiments. As a rule, any embodiment referenced herein is freely combinable with any one or more of the other embodiments referenced herein, and any number of features of different embodiments are combinable with one another, unless indicated otherwise.

Although what has been described in detail here is with reference to certain embodiments, one skilled in the art will appreciate that the present invention can be practiced by someone other than with the described embodiments, which have been presented for purposes of illustration and not of limitation. Therefore, the scope of the appended claims should not be limited to the description of the embodiments contained herein.

1. A prosthetic system for providing motion between a first bone and a second bone of a joint of a subject, the first bone having a metaphyseal zone and a diaphyseal zone, the system comprising:
a support structure for use in implanting a component of an implant in the first bone of the subject, the support structure having a first end surface, a second end surface, an exterior surface extending from the first end surface to the second end surface, and an inner surface defining a passageway extending from the first end surface to the second end surface,
wherein the exterior surface of the support structure is configured to be received in a cavity of the first bone such that the first end surface is flush with or beneath a surface of the first bone, and
wherein the exterior surface of the support structure is configured to be received in the cavity of the first bone such that a section of the exterior surface of the support structure contacts cortical bone in the diaphyseal zone of the first bone.

2. The prosthetic system of claim 1 wherein:
the component includes a body and a stem extending away from the body, and
the stem is positioned within the passageway of the support structure.

3. The prosthetic system of claim 1 wherein:
the first bone is the tibia, and
the joint is the knee.

4. The prosthetic system of claim 3 wherein:
the component comprises a tibial component having a body and a stem extending away from the body,
wherein the stem is positioned within the passageway of the support structure.

5. The prosthetic system of claim 4 wherein:
the body of the tibial component is a tibial tray.

6. The prosthetic system of claim 1 wherein:
the first bone is the femur, and
the joint is the knee.

7. The prosthetic system of claim 6 wherein:
the component comprises a femoral component having medial and lateral condyles with curved articulating surfaces.

8. The prosthetic system of claim 1 wherein:
the exterior surface of the support structure is roughened.

9. The prosthetic system of claim 1 wherein:
the exterior surface of the support structure comprises a porous ingrowth material.

10. The prosthetic system of claim 1 wherein:
the support structure has a wall between the exterior surface and the inner surface, and
the wall includes a slot extending away from the first end surface.

11. The prosthetic system of claim 10 wherein:
the component includes a body and a stem extending away from the body, and
the stem is positioned within the passageway of the support structure such that a keel of the component is positioned within the slot.

12. The prosthetic system of claim 1 wherein:
the support structure has a wall between the exterior surface and the inner surface, and
the wall includes a pair of slots extending away from the first end surface.

13. The prosthetic system of claim 12 wherein:
the component includes a body and a stem extending away from the body, and
the stem is positioned within the passageway of the support structure such that a first keel of the component is positioned within one of the pair of slots and a second keel of the component is positioned within another of the pair of slots.

14. The prosthetic system of claim 1 wherein:
the component includes a body and a stem extending away from the body, and
the stem is positioned within the passageway of the support structure such that an end of the stem opposite the body extends beyond the second end surface of the support structure.

15. The prosthetic system of claim 1 wherein:
a portion of the exterior surface of the support structure has a frustoconical shape.

16. The prosthetic system of claim 1 wherein:
an entirety of the exterior surface of the support structure has a frustoconical shape.

17. The prosthetic system of claim 1 wherein:
a longitudinal length of the support structure from the first end surface to the second end surface is in a range of 65 millimeters to 105 millimeters.

18. The prosthetic system of claim 1 wherein:
a transverse length of an outside diameter at the first end surface of the support structure is in a range of 25 millimeters to 45 millimeters.

19. The prosthetic system of claim 1 wherein:
a transverse length of an outside diameter at the second end surface of the support structure is in a range of 15 millimeters to 27 millimeters.

20. The prosthetic system of claim 1 wherein:
the exterior surface of the support structure is configured to be received in the cavity of the first bone such that another section of the exterior surface of the support structure contacts cortical bone in the metaphyseal zone of the first bone.

21. The prosthetic system of claim 1 wherein:
the first end surface is within an axial plane defined by an outermost edge of the first end surface, and
a longitudinal axis of the passageway of the support structure is offset with respect to a geometric center point of the axial plane.

22. A prosthetic system for providing motion between a first bone and a second bone of a joint of a subject, the first bone having a metaphyseal zone and a diaphyseal zone, the system comprising:
a support structure for use in implanting a component of an implant in the first bone of the subject, the support structure having a first end surface, a second end surface, an exterior surface extending from the first end surface to the second end surface, and an inner surface defining a passageway extending from the first end surface to the second end surface,
wherein the exterior surface of the support structure is configured to be received in a cavity of the first bone such that the first end surface is flush with or beneath a surface of the first bone,
wherein the exterior surface of the support structure is configured to be received in the cavity of the first bone such that a section of the exterior surface of the support structure contacts cortical bone in the diaphyseal zone of the first bone, and
wherein the support structure has bilateral symmetry with respect to a longitudinal plane containing a longitudinal axis of the passageway.

23. The prosthetic system of claim 22 wherein:
a portion of the exterior surface of the support structure has a frustoconical shape.

24. The prosthetic system of claim 22 wherein:
an entirety of the exterior surface of the support structure has a frustoconical shape.

25. A prosthetic system for providing motion between a first bone and a second bone of a joint of a subject, the first bone having a metaphyseal zone and a diaphyseal zone, the system comprising:
a support structure for use in implanting a component of an implant in the first bone of the subject, the support structure having a first end surface, a second end surface, an exterior surface extending from the first end surface to the second end surface, and an inner surface defining a passageway extending from the first end surface to the second end surface,
wherein the exterior surface of the support structure is configured to be received in a cavity of the first bone such that the first end surface is flush with or beneath a surface of the first bone,
wherein the exterior surface of the support structure is configured to be received in the cavity of the first bone such that a section of the exterior surface of the support structure contacts cortical bone in the diaphyseal zone of the first bone, and
the support structure does not have bilateral symmetry with respect to a longitudinal plane containing a longitudinal axis of the passageway.

26. The prosthetic system of claim 25 wherein:
the support structure has a wall between the exterior surface and the inner surface, and
the wall includes opposed lobes that terminate at the first end surface of the support structure.

27. The prosthetic system of claim 26 wherein:
one of the opposed lobes extends a first distance away from a longitudinal plane containing a longitudinal axis of the passageway,
the other of the opposed lobes extends a second distance away from the longitudinal plane containing the longitudinal axis of the passageway, and
the first distance is greater than the second distance.

28. The prosthetic system of claim 27 wherein:
the first distance is in a range of 22 millimeters to 40 millimeters.

29. The prosthetic system of claim 27 wherein:
the second distance is in a range of 18 millimeters to 35 millimeters.

30. An apparatus for fabricating a support structure for use in implanting a component of an implant in a first bone of a subject, the first bone having a metaphyseal zone and a diaphyseal zone, the apparatus comprising:
a 3D printing device; and
a controller in electrical communication with the 3D printing device, the controller being configured to execute a program stored in the controller to:
(i) receive a 3D digital model of the support structure to be fabricated wherein the support structure has a first end surface, a second end surface, an exterior surface extending from the first end surface to the second end surface, and an inner surface defining a passageway extending from the first end surface to the second end surface, wherein the exterior surface of the support structure is configured to be received in a cavity of the first bone such that the first end surface is flush with or beneath a surface of the first bone, and wherein the exterior surface of the support structure is configured to be received in the cavity of the first bone such that a section of the exterior surface of the support structure contacts cortical bone in the diaphyseal zone of the first bone; and
(ii) send a group of signals to the 3D printing device to fabricate the support structure from the 3D digital model.
